(19) United States
(12) Patent Application Publication
(10) Pub. No.: US 2024/0374811 A1
(43) Pub. Date: Nov. 14, 2024

(54) INFUSION DEVICE AUTOMATED PROGRAMMING MITIGATION

(71) Applicant: CareFusion 303, Inc., San Diego, CA (US)

(72) Inventor: Michael K. WORKMAN, Carlsbad, CA (US)

(21) Appl. No.: 18/692,781

(22) PCT Filed: Sep. 19, 2022
(86) PCT No.: PCT/US2022/044027

§ 371 Date: Mar. 15, 2024

Related U.S. Application Data
(60) Provisional application No. 63/246,270, filed on Sep. 20, 2021.

(51) Int. Cl. A61M 5/142; A61M 5/14; G16H 40/63; G16H 40/67; G16H 70/40

(52) U.S. Cl. CPC A61M 5/142; A61M 5/1407; A61M 5/1413; G16H 40/63; G16H 40/67; G16H 70/40; A61M 2005/14208; A61M 2205/3553; A61M 2205/502

(57) ABSTRACT

When automated programming data configured to instruct an infusion device regarding an infusion of a medication is received from a server, the disclosed system and method identifies a deviation in the data with respect to stored programming data. Without rejecting the data for the deviation, the infusion device instead sends an acknowledgement of the automated programming data and the deviation, and determines a user interface workflow for correcting the deviation. The infusion device then provides for display one or more user prompts according to the determined user interface workflow to resolve the deviation. If the deviation is resolved by way of user responses, then the infusion is activated. Otherwise, an indication is sent to the server regarding the deviation.

### TECHNICAL FIELD

This application relates generally to ensuring that an infusion device is properly programmed.

### BACKGROUND

Modern infusion pumps provide, via a user interface, manual workflows with regard to programming infusion. In this regard, an infusion pump may receive infusion order parameters from a third-party system to configure the pump to deliver a specific fluid, at specific rates, for a specific patient, a process which is often referred to as an automated programming request (APR). The configuration may include pre-populating values for parameters presented via the user interface.

Pre-population of infusion parameters reduces the number of programming screens, key presses, and potential input errors that may exist with manual programming. Generally, there may be no differences in programming screens, prompts or the user interface between systems configured for APR and those that are not, and the implementation of APRs does not preclude a clinician from manually programming the pump. However, manual programming is required in the event of a failure in any component of the interfaced system.

In some APR enabled systems, APR orders may be rejected due to device to order deviations that prevent the system from taking automatic action based on the order. For example, an infusion device may be configured to not infuse at a flow rate greater than 999 mL/h. If the APR order includes a rate parameter value of 1000 mL/h, then the order may be rejected because the auto-programmed parameter value is misaligned with the pump configuration. When an order is rejected, the device must be programmed manually to initiate the infusion.

### SUMMARY

The subject technology provides a mechanism and corresponding algorithm that identifies common non clinically significant APR misalignment errors in APR orders. Unlike legacy systems, the infusion devices employing the subject technology accept misaligned APRs in real time and continue to process to resolution with a clinician any misalignment on a local user interface of the device. Accordingly, the infusion device accepts “good” data while filtering out the misaligned data to be manually entered by the clinician. The infusion device reports misalignment issues to a server and, in some implementations, utilizes machine learning to learn new associations as to avoid flagging misalignment in future APR orders.

In this regard, the subject technology includes an infusion device, comprising: a display device; and a pump controller comprising a processor, wherein the pump controller is configured to: receive, from a server remote from the infusion device, automated programming data configured to instruct the infusion device regarding an infusion of a medication; identify a deviation in the received automated programming data with respect to stored programming data; send, without rejecting the received automated programming data, an acknowledgement of the automated programming data and the deviation; determining a user interface workflow for correcting the deviation; provide, via a user interface displayed on the display device, one or more user prompts according to the determined user interface workflow to resolved the deviation; determine whether the deviation has been resolved; if the deviation is resolved then activate the infusion; and if the deviation is not resolved then send an indication to the server regarding the deviation. Other aspects include corresponding systems, methods, and computer program products for implementation of the corresponding device and its features.

The subject technology also relates to a method for mitigating automated programming request errors in a medical device. The method, performed by an infusion device, comprises: receiving, from a server remote from the infusion device, automated programming data configured to instruct the infusion device regarding an infusion of a medication; identifying a deviation in the received automated programming data with respect to stored programming data; sending, without rejecting the received automated programming data, an acknowledgement of the automated programming data and the deviation; determining a user interface workflow for correcting the deviation; providing, via a user interface displayed on a display of the infusion device, one or more user prompts according to the determined user interface workflow to resolved the deviation; determining whether the deviation has been resolved; if the deviation is resolved then activating the infusion; and if the deviation is not resolved then sending an indication to the server regarding the deviation. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example patient care system that includes an infusion device.

FIG. 1B depicts a closer view of a portion of the patient care system shown in FIG. 1A.

FIG. 1C depicts an example of an institutional patient care system of a healthcare organization, according to aspects of the subject technology.

FIG. 2 depicts a first example process for mitigating automated programming request errors in a medical device, according to aspects of the subject technology.

FIG. 3 depicts a second example process for mitigating automated programming request errors in a medical device, according to aspects of the subject technology.

FIG. 4 is a conceptual diagram illustrating an example electronic system for mitigating automated programming request errors in a medical device, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

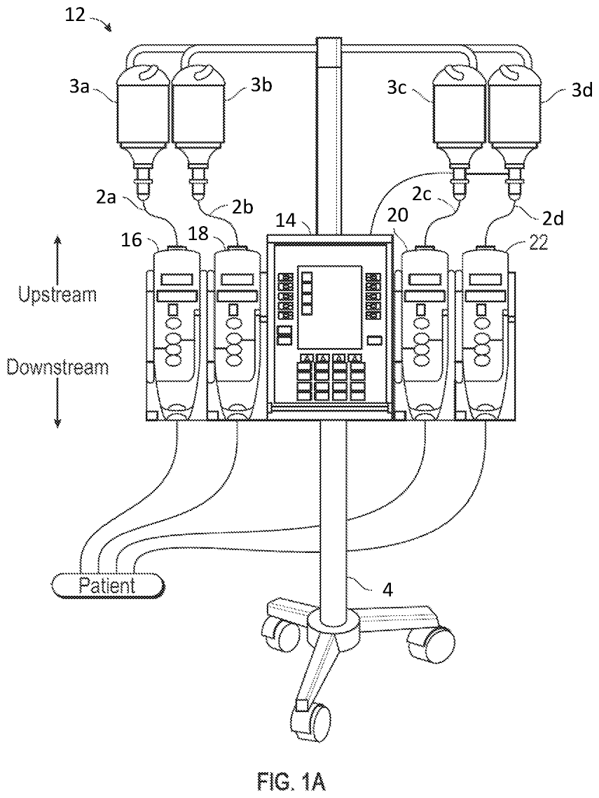

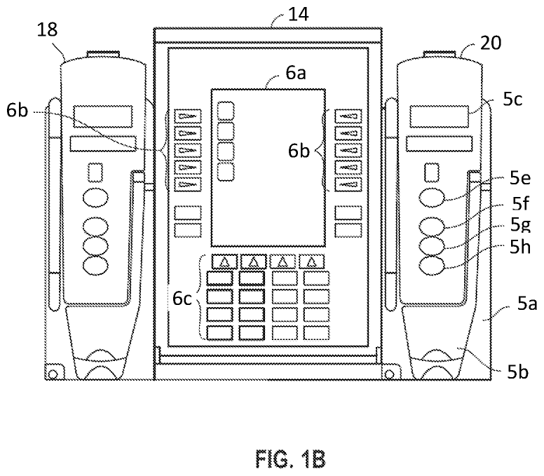

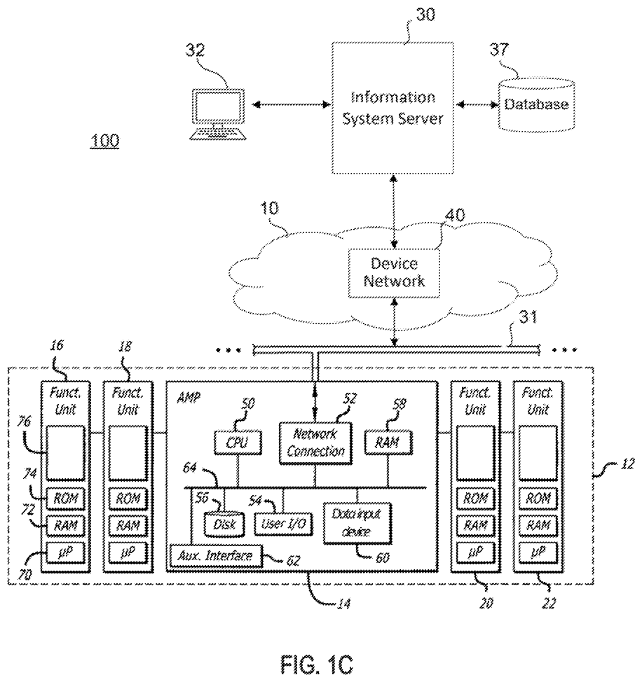

FIG. 1A is an example patient care system, according to various aspects of the subject technology. The patient care system 1 shown in FIG. 1A includes four fluid infusion pumps 16, 18, 20, 22, each of which is in operative engagement with a respective fluid administration set 2a-d. Fluid supplies 3a-d, which may take various forms but in this case are shown as bottles, are inverted and suspended above the pumps. Fluid supplies may also take the form of bags or other types of containers. Both the patient care system 1 and the fluid supplies 3a-d are mounted to a roller stand or pole 4. The specific fluid supplies as well as their orientation (e.g., mount location, mount height, mounting type, etc.) within the care area may generate one or more interaction records. The interaction record for a set for example may be generated in part by detecting a scannable code associated with the set or detecting a physical structure on the set that encodes identifying information for the set prior to use.

As shown in the example implementation of FIG. 1A, each administration set 2a-d is connected between a respective fluid supply 3a-d and the same patient so that the patient may receive the fluids in all the fluid supplies. The administration set may be identified either actively by, for example, scanning by a clinician or passively by, for example, wireless or optical detection of the administration set.

In the depicted example, a separate infusion pump 16, 18, 20, 22 is used to infuse each of the fluids of the fluid supplies into the patient. The infusion pumps are flow control devices that will act on the respective tube or fluid conduit of the fluid administration set to move the fluid from the fluid supply through the conduit to the patient. Because individual pumps are used, each may be individually set to the pumping or operating parameters required for infusing the particular medical fluid from the respective fluid supply into the patient at the particular rate prescribed for that fluid by the clinician.

Typically, medical fluid administration sets have more parts than are shown in FIG. 1A. Many have check valves, drip chambers, valved ports, connectors, and other devices well known to those skilled in the art. These other devices have not been included in the drawings so as to preserve clarity of illustration.

FIG. 1B is a closer view of a portion of the example patient care system shown in FIG. 1A, according to various aspects of the subject technology. FIG. 1B shows two of the fluid infusion pumps mounted at either side of a programming module, and the displays and control keys of each, with the programming module being capable of programming both infusion pumps. The infusion device 12 includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages). Control keys 5e-h exist for programming and controlling operations of the infusion pump as desired. In some implementations, the control keys may be presented as interactive elements on the display 5c (e.g., touchscreen display). The infusion device 12 and/or infusion pump 20 may also include audio alert equipment in the form of a speaker (not shown).

The programming module 14 of the infusion device 12 includes a display 6a for visually communicating various information, such as the operating parameters of a connected pump and alert indications and alert messages. The programming module 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. The programming module 14 may include a communications system (not shown) with which the programming module 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). The communication module may be used to transfer access and interaction information for clinicians encountering the programming module or device coupled therewith (e.g., pump 20 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the programming module 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

In some embodiments, the pressure measurements from the upstream and/or downstream pressure sensors are transmitted to a server or other coordination device, and the methods disclosed herein are implemented on the server or other coordination device. For example, more sophisticated and computationally intensive approaches like machine-learning can be implemented on the server (or on a PCU with a larger memory and/or CPU resources). In some embodiments, machine learning is used to identify syringe pump empty conditions based on pressure signals received from the pump.

FIG. 1C depicts an example of an institutional patient care system 100 of a healthcare organization, according to aspects of the subject technology. In FIG. 1C, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1C optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 130. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, and mobile devices such as laptops, tablet computers, augmented reality devices, or smartphones, configured with software for communications with information system server 130 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1C to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 116, 118, 120 and 122, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1C, at least one of functional modules 16, 18, 20, 22 may be an infusion pump module such as an intravenous infusion pump for delivering medication or other fluid to a patient. For the purposes of this discussion, functional module 116 is an infusion pump module. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may be a printer, scanner, bar code reader, near-field communication reader, RFID reader, or any other peripheral input, output or input/output device.

Each functional module 16, 18, 20 and/or 22 communicates directly or indirectly with interface unit 14, with interface unit 14 providing overall monitoring and control of device 12. Functional modules 16, 18, 20 and/or 22 may be connected physically and electronically in serial fashion to one or both ends of interface unit 14 as shown in FIG. 1C, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1C, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1C), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information system (HIS) server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

With further reference to FIG. 1C, patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the interface unit 14 may contain the drug library or libraries, an event log or logs, and pump configuration settings, such as, but not limited to, profiles to be used in particular practice areas such as ICU, PED, etc. The memory may be electronically loadable memory such as non-volatile memory (e.g. EEPROM). Drug libraries, which illustratively contain such information as the drug names, ranges of delivery parameter values such as proper concentrations, dosage units, and dose limits, and can be used to perform drug calculation-based infusions.

The drug library stored within the pump's memory may include limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

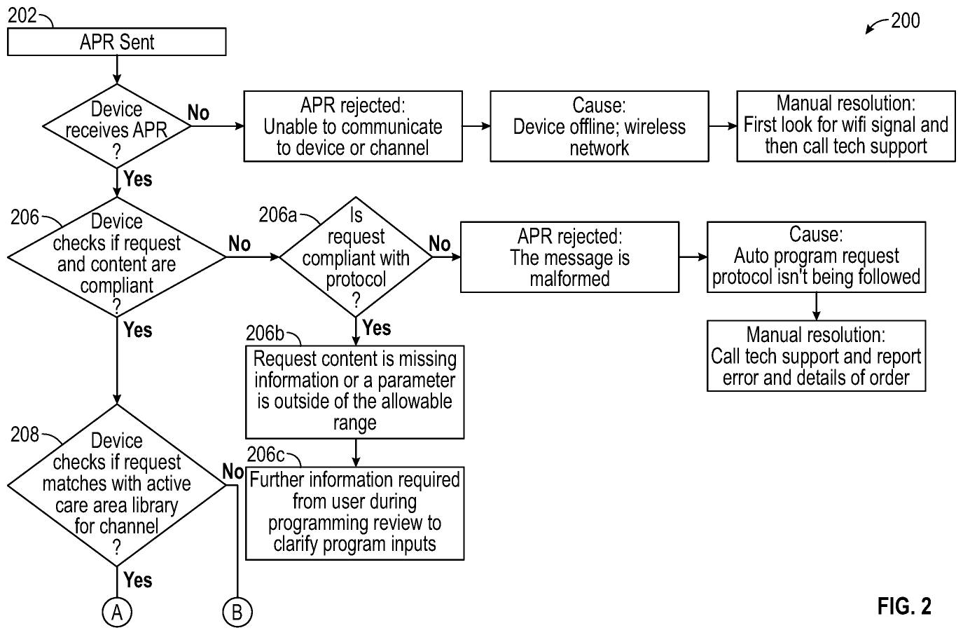

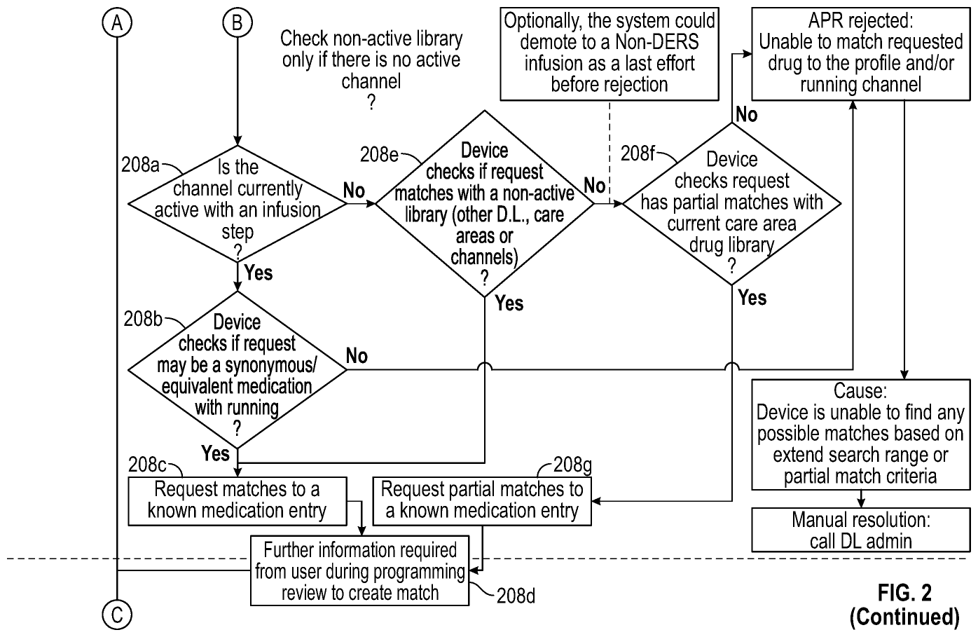

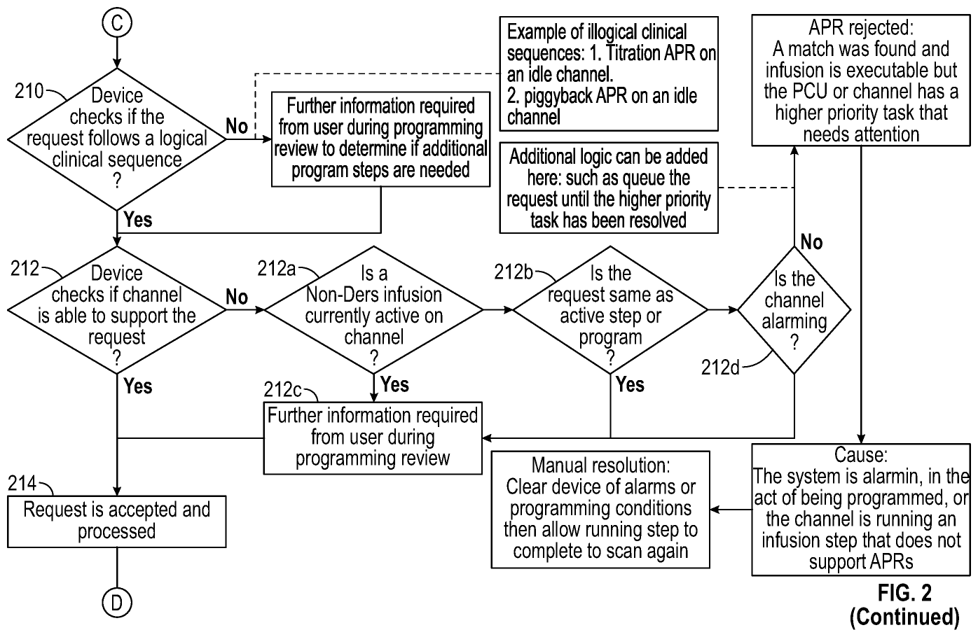

FIG. 2 depicts a first example process for mitigating automated programming request errors in a medical device, according to aspects of the subject technology. Typically, an automated programming request is sent (202) to a medical device as a result of an initial action taken at the medical device. For example, a clinician may scan a medical item using a scanner associated with the medical device. The scanner is not required to be integrated with the medical device. The scanner may be part of a separate device (e.g., part of one or more computing devices) connected to the same network 40 as the medical device and configured with software to function in an overall workflow involving the medical device. The scanning initiates a process by which information pertaining to the item (e.g., scanned from a code affixed to or transmitted by the item) is automatically sent to a centralized server 30 via a network 40. The server 30 performs certain actions pertaining to the item and sends a request to the medical device 12 to load parameters pertaining to the item. The parameters may be stored in the medical device 12, but loaded in response to an identifier received from the server. While the depicted examples involve an infusion device, any medical device may be configured in the same or similar manner and employ the automated programming error mitigation described herein.

According to some implementations, the infusion device 12 determines whether the APR and content are compliant (206). The compliance determination may be based on the data fields included in the APR. For example, the APR may include or omit a field that may prevent the pump from parsing or auto-programming the pump using the order alone. The compliance determination may be based on values included within a data field of the APR. For example, if the specified value for a parameter is outside the configured range of the infusion pump, the value may be determined to be non-compliant.

Determining whether the request is compliant may include determining whether it is compliant with a protocol understandable by the pump (206a). If not compliant with the protocol, then the request may be rejected in part because the pump cannot process the request. If the APR is compliant with an understandable protocol, then the APR may be at least preliminarily accepted, and an acknowledgement sent from the infusion device to the server 30 indicating that the APR is being processed. The system may then proceed to determine, as further described below, whether the APR includes any deviations.

The APR may include an identifier and/or name for the drug to use for a particular order. If the drug is not found in the current drug library, the pump is configured to search other drug libraries stored within the pump's memory for the drug. The pump may search based on the name of the drug or based on certain parameters provided in the APR. The server may send, for example, the drug name, the primary identifier or alias (drug id), and a concentration. So, the pump may transmit a search request based on one or more of these parameters.

In some implementations, the infusion device 12 (and/or processor or system) determines whether information in the request matches known parameters for the corresponding match in the drug library (206b). For example, if the APR includes a certain drug concentration, the infusion device may check the drug library to determine whether the drug concentration received is within an allowable range for the drug identified by the APR. If the concentration (or other parameter) is outside of the allowable range (e.g., clinically defined dose error reduction range), then the system may prompt via the display of the infusion device (see FIG. 1B) for a user to update or confirm the concentration (206c). On receiving the user input, the process may continue, including loading the remaining parameters for the infusion designated by the APR and activating the infusion. In some implementations, the user input request may be accumulated along with other information.

A patient may change care areas, but the pump may still be set up for the former care area. For example, the patient may be switched from critical care to the operating room. A drug may be sent for an operating room, but the patient is still in critical care. The drug may not be in the critical care area drug library. In this regard, the subject technology may check the system for safety rules defined for other care areas for the same drug.

In this regard, the system may perform different functions based on whether an infusion is actively being administered by the infusion device (208a). Active may include providing a fluid at a specified rate. In some implementations, the system may determine whether the medication identified in the APR is synonymous or equivalent with the medication currently being administered (208b). If the identified medication is synonymous or matches with a non-active library (208c) then the system may prompt the user for confirmation to merge the APR with the active infusion and/or provide further information to confirm and/or create a match (208d).

Drug libraries may be stored and/or accessed or indexed within the memory of the infusion device based on care area. If there is no active channel synonymous with the APR then the system may determine whether the APR matches with information in a drug library (208e). A drug library loaded for a current care area may be searched to determine whether it supports the medication identified in the APR. If the drug library(s) for the current care area does not match the care area corresponding to the APR, or the drug identified by the APR is not in the drug library(s) stored for a current care area then the system may check within drug libraries of other care areas. If a match is found (208c) then the system may prompt the user for confirmation to confirm and/or create the match (208d).

If no match is found then the system may make a determination of whether the information within the APR is a partial match with information in a drug library (208f). If there is a partial match (208g) then further information may be requested to confirm and/or create a match (208d). In some implementations, if the drug is not found in other drug libraries within the pump's memory, the pump may send a request to the server for a data set for the drug. The server may then send down a specific dataset for the drug, including guardrails and parameters for the drug. The pump may then store the drug in the current drug library, or may temporarily store the parameters for use during the current session and/or while the patient is in the current care area.

In some implementations, the information of the APR may be analyzed to determine whether the information is part of a logical clinical sequence (210). For example, the server 30 may send down an APR request for a bolus delivery to a pump channel that currently does not have an infusion on it at all, or to a channel that is idle. If there is no infusion running, then the device 12 cannot perform the bolus. Consequently, the clinical sequence requested may not be in a proper order. The system may prompt the user for further information. For example, when the APR is related to an ongoing infusion, and the infusion is not currently active, the system may prompt the clinician to start or continue administering the infusion to the patient.

In another example, the server may send down a secondary order, and the infusion device has not loaded a primary order for the patient. In this example, the pump may immediately switch the interface to begin programming of the primary order, or to request the primary order from the server. Once the primary order is loaded then the secondary order, already received from the server will be loaded.

In some implementations, the information of the APR may be analyzed to determine whether a channel (or the active channel) is able to support the APR (212). The system may determine whether there is a manually programmed (e.g., not based on an APR) infusion running on the current channel (212a). If a current program has been input or is in the process of being input then the system may prompt the user for confirmation to confirm use of the APR or merge the data therein (212c). In another example, a clinician may begin programming the infusion pump for an infusion on a particular pump channel, but stops and requests an APR instead. Accordingly, the device is already in a state of being programmed. On receiving the APR, the system may identify the incomplete program and prompt the clinician to confirm whether the new APR should be aligned with or take the place of the incomplete program.

As a further example within some implementations, the clinician may have entered a drug identifier and/or other information for the drug, or may select a drug from a currently enabled drug library. The clinician may then realize that obtaining an APR from the server would be more efficient. The clinician scans a medication bag, which then initiates an APR from the server. The pump receives the APR and matches the drug identifier in the APR (or other information) with the information currently being programmed into the pump. The infusion pump may then prompt the clinician to confirm that the APR data should be merged with the session record currently being programmed (212b). At this point the clinician may confirm that the data should be merged (e.g., fill in the missing data) and the pump may continue based on the record started by the clinician (now complete with additional data from the server); or the clinician may select to receive the APR and dismiss the current programming (212c). In some implementations, the APR may erase and replace the data of the record. Additionally, or in the alternative, if another channel is available, the clinician may be presented with the option to receive the APR on the alternate channel. In another example, the channel may be in an alarm state when the APR is received (212d). The system may prompt the user to dismiss the alarm before the APR is accepted.

In some implementations, the clinician may scan a medication for a first pump channel; however, the channel may not be compatible with the scanned medication. For example, the clinician may scan a syringe for a syringe pump using a channel for a peristaltic pump. When the APR is received by the infusion device, the system may identify the discontinuity and perform a search to determine whether the drug is in another drug library and/or the correct channel is available. For example, the system may determine that the drug is available in a syringe drug library and that a syringe pump is connected, notify the clinician that the APR data is not compatible with the currently selected channel, and prompt the clinician to confirm whether the channel hosting the syringe pump should be used. If the syringe pump is already in use, then the system may prompt the clinician to confirm whether the new APR data should be classified as a secondary order.

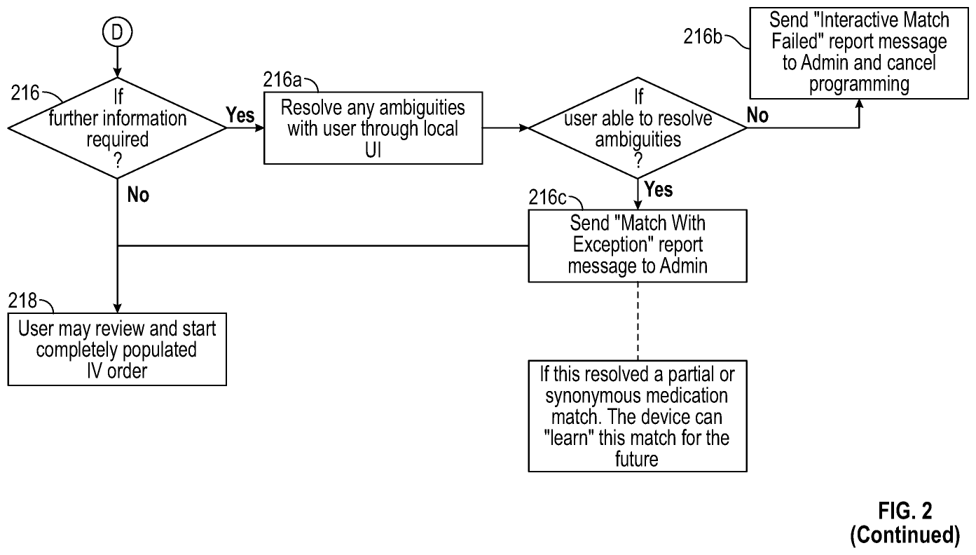

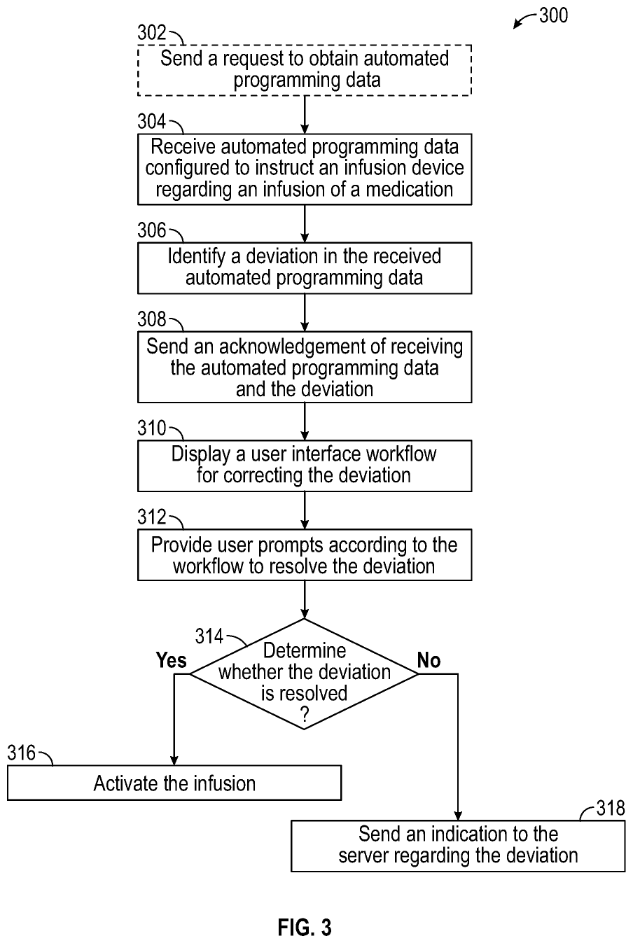

After the foregoing determinations have been made and any issues of been resolved, the infusion device may send confirmation to the server that the APR was accepted and processed (214). In some implementations, the foregoing checks are performed on the APR when it is received and, if there are any deviations (e.g., from information within stored drug libraries) then the system may send its acknowledgement of receiving the APR with an indication that there is deviation that will require resolution by the clinician. In this manner, the process continues for real time resolution by the clinician without failure of the APR. The system may then attempt to resolve the deviation by way of prompting the clinician according to workflow of FIG. 3 and activate the infusion upon resolving the deviation(s). If the deviation is not resolved then the system may send an indication to the server regarding the deviation (e.g., an interactive match failed report message). This indication may include a report of the deviation.

In some implementations, further information may be requested from the user regarding the current programming (216). In some implementations, such information may be regarding ambiguities in the data, or may be minor ancillary reporting input requested for notation of the patient's records (216a). In some instances, failure to include the information may cause a failure message to be sent to the server and/or cancel programming the device (216b). Provided the remaining ambiguities are minor and/or resolved by the user through the user interface, the user may then review the final programming and start the infusion (218). A message may be sent to the server indicating that the APR was accepted and implemented, but with a resolved deviation (216c). In some implementations, the infusion device 12 may be locked by the system from performing an infusion pertaining to the order received in the APR until all deviations and/or ambiguities are resolved.

FIG. 3 depicts a second example process for mitigating automated programming request errors in a medical device, according to aspects of the subject technology. For explanatory purposes, the various blocks of example process 300 are described herein with reference to FIGS. 1A, 1B, 1C, and 2, and the components and/or processes described herein. The one or more of the blocks of process 300 may be implemented, for example, by one or more computing devices including, for example, medical device 12. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, a request to obtain automated programming data (e.g., an APR) is optionally sent from the device 12 to the server 30 (302). In some implementations, the server 30, having been in communication with the device 12, may send the programming data without a request. One example of how the request to obtain the automated programming data may include, for example, the system (or a supporting system) receiving scanned information from a medication container that was scanned by a scanner associated with the infusion device, and sending the scanned information to the server with a request to transmit the automated programming data to the infusion device.

The infusion device 12 receives, from the server 30, automated programming data configured to instruct the infusion device regarding an infusion of a medication (e.g., the scanned medication) (304). The automated programming data may be in the form of a programming request and may include, for example, a drug identifier and a request to load parameters for a drug corresponding to the drug identifier. As described above, an APR may instruct the infusion device to load the parameters from an available drug library stored on the infusion device. According to various implementations, the APR may include a concentration for the drug.

The infusion device identifies a deviation in the received automated programming data with respect to stored programming data (306). In some implementations, the deviation arises from a drug identified in the APR not being identified in a currently active first drug library stored in a memory of the infusion device 12. In some implementations, the deviation may include the received concentration of the drug being outside an allowable range for the concentration stored in the drug library for the drug.

In some implementations, the system may determine that the parameters to be loaded for drug are stored in a deactivated second drug library stored in the memory of the infusion pump. In some implementations, the system may determine that the second drug library is for a different care area than a care area currently activated for the infusion device.

In some implementations, the deviation may include receiving an APR for changing a parameter of the infusion of the medication while the infusion is being administered to a patient but determining, when the automated programming data is received, that the infusion is not being administered to the patient.

In some implementations, the infusion device includes a first pump channel and a second pump channel (e.g., corresponding to multiple infusion modules 18, 20). The system may identify the first pump channel for receiving the automated programming data and determine that the parameters to be loaded are more aligned with the second pump channel than with the first pump channel. In some implementations, the deviation may include the automated programming data being for a drug to be administered after a first drug is administered to the patient, but the first drug is not currently being administered.

An acknowledgement of the infusion device 12 receiving the automated programming data and of the deviation may be sent (308). In some implementations, the acknowledgement may be sent when the programming data is determined to be compliant with protocol. In some implementations, the system performs a real time check on the data with regard to data in locally stored drug libraries to determine whether a deviation exists prior to the acknowledgement. If the deviation exists, then the acknowledgment may be sent before the deviation is resolved. In this manner, the infusion device may accept the deviation with the intent to resolve the deviation through user interaction.

The infusion device (or related system) displays a user interface workflow for resolving the deviation (310) in accordance with the processes of FIG. 2. The user interface workflow may involve steps and/or user input required to resolve the deviation according to stored patterns in the device 12. In this regard, the workflow may be modeled based on the process flow depicted in FIG. 2. In some implementations, the workflow may be generated and stored by a machine learning algorithm over time based on training data.

When a deviation exists, a user interface is provided (e.g., displayed on a display device of the infusion device) to resolve the deviation, including one or more user prompts according to the determined user interface workflow (312). To resolve a care area deviation, the system may prompt via the one or more prompts, a user to confirm use of the second drug library. Based on receiving a confirmation to use the second drug library, the system may then deactivate the currently active first drug library, activate the second drug library, and load the parameters for the drug from the activated second drug library. To resolve an incorrect channel deviation, the system may then prompt via the one or more prompts, a user to confirm use of the second channel for receiving the automated programming data.

In some implementations, wherein the automated programming data is received after scanned information is sent to the server (e.g., from scanning a medication), the system may determine that the infusion device 12 already received at least some parameters for the medication (e.g., via the user interface) and prompt via the one or more prompts, a user to confirm use of the automated programming data received from the server in place of the at least some parameters already received. In some implementations, when an infusion identified by the APR is not being administered, the system may prompt, via the one or more user prompts, a user to start or continue administering the infusion to the patient.

If the automated programming data is for a drug to be administered to a patient after a first drug is administered to the patient, but the first drug is not currently being administered (e.g., not active on any channel), then the system may prompt via the one or more prompts, a user to program the infusion device to administer the first drug. If the automated programming data was sent for an inactive infusion that was resolved by the user programming and/or activating the infusion, the system may automatically load parameters corresponding to the automated programming data from the stored programming data upon the resolution.

In some implementations, if the drug is not found in other drug libraries within the pump's memory, the infusion device 12 may send a request to the server 30 for a data set for the drug. The server may then send down a specific dataset for the drug, including guardrails and parameters for the drug. In some implementations, the user may be prompted confirm the download. The infusion device 12 may then store the drug in the current drug library, or may temporarily store the parameters for use during the current session and/or while the patient is in the current care area.

Continuing with process 300, a determination is made as to whether the deviation has been resolved (314). If the deviation is resolved, then the infusion is activated (316). The activation may be based on some parameters and values from the APR and any deviations corrected or clarified such as through the user interface or queries of data stored in a memory (e.g., drug libraries for other care areas). Once activated, the infusion device 12 may transmit a message to the server 30 such as a message indicating that the APR was accepted or that infusion has started. If the deviation is not resolved, the infusion device 12 may send an indication to the server regarding the deviation (318). For example, an interactive match failed report message may be sent to the server 30. The report may identify the parameter or value that was not resolved. In some implementations, the report may include a rule or library entry that was used to determine compliance. Based on this information the system may further identify configuration errors based on, for example, an aggregated review of unresolved APR discrepancies.

According to various implementations, the system may involve machine learning. For example, the system may resolve a plurality of deviations for a plurality of automated programming data received from the server and identify a common resolution to a common deviation of the plurality of deviations based on a plurality of user responses to user prompts for resolving the deviations. The system may then, at some later point, receive, by the infusion device, new automated programming data for a new infusion to be administered by the infusion device, identify the common deviation in the new automated programming data, and automatically resolve the common deviation in the new automated programming data based on the identified common resolution.

The foregoing process provides multiple benefits including, but not limited to, ensuring the patient receives all of the prescribed medicine. Moreover, early detection of a syringe being fully emptied reduces strain on the pump thereby conserving the resources needed to deliver the fluid such as power, pumping motor cycles, and pumping finger wear. Indeed, many infusion pumps are battery powered and power limited. In such power constrained systems, the life of a battery may be extended by preventing an infusion from running needlessly when no fluid is being pumped. The system detects the threshold trigger condition and initiates rapid emptying of the syringe or other remedial remedies (such as reducing an end of delivery threshold) to ensure the infusion is timely completed.

Many of the above-described example process 300, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

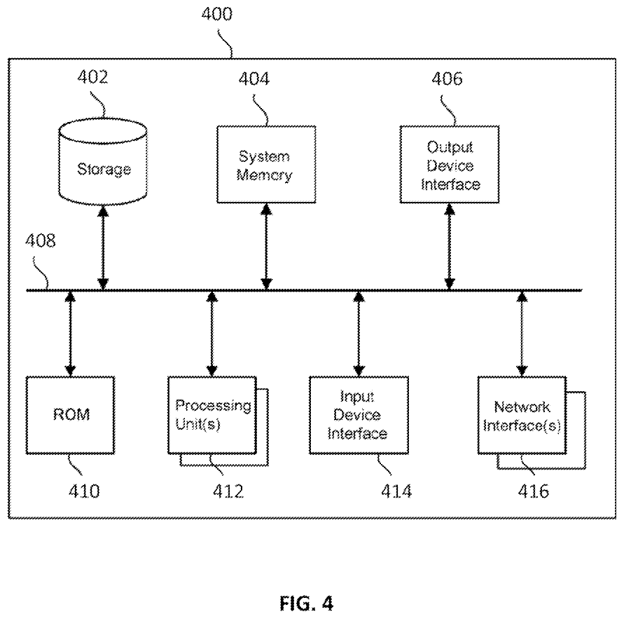

FIG. 4 is a conceptual diagram illustrating an example electronic system 400 for mitigating automated programming request errors in a medical device, according to aspects of the subject technology. Electronic system 400 may be a computing device for execution of software associated with one or more portions or steps of processes 200, 300, or components and methods provided by FIGS. 1-3, including but not limited to computing hardware within patient care device 12, and/or any computing devices or associated terminals disclosed herein. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 414, an output device interface 406, and one or more network interfaces 416. In some implementations, electronic system 400 may include or be integrated with other computing devices or circuitry for operation of the various components and methods previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process, in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such as, random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. An infusion device, comprising: a display device; and a pump controller comprising a processor; wherein the pump controller is configured to: receive, from a server remote from the infusion device, automated programming data configured to instruct the infusion device regarding an infusion of a medication; identify a deviation in the received automated programming data with respect to stored programming data; send, without rejecting the received automated programming data, an acknowledgement of the automated programming data and the deviation; display a user interface workflow for correcting the deviation; provide, via a user interface displayed on the display device, one or more user prompts according to the determined user interface workflow to resolved the deviation; determine whether the deviation has been resolved; if the deviation is resolved then activate the infusion; and if the deviation is not resolved then send an indication to the server regarding the deviation.

Clause 2. The infusion device of Clause 1, wherein receiving the automated programming data comprises receiving a drug identifier and a request to load parameters for a drug corresponding to the drug identifier; and wherein identifying the deviation comprises determining that the drug is not identified in a currently active first drug library stored in a memory of the infusion device.

Clause 3. The infusion device of Clause 2, wherein the pump controller is further configured to: determine, by the infusion device, that the parameters to be loaded for drug are stored in a deactivated second drug library stored in the memory of the infusion device; and prompt via the one or more prompts, a user to confirm use of the second drug library; and based on receiving a confirmation to use the second drug library, deactivate the currently active first drug library, activate the second drug library, and load the parameters for the drug from the activated second drug library.

Clause 4. The infusion device of Clause 3, wherein the infusion device comprises a first pump channel and a second pump channel, wherein the pump controller is further configured to: identify the first pump channel for receiving the automated programming data; determine that the parameters to be loaded are more aligned with the second pump channel than with the first pump channel; and prompt via the one or more prompts, a user to confirm use of the second pump channel for receiving the automated programming data.

Clause 5. The infusion device of Clause 3 or Clause 4, wherein the pump controller is further configured to: determine that the second drug library is for a different care area than a care area currently activated for the infusion device.

Clause 6. The infusion device of any one of Clauses 1 through 5, wherein receiving the automated programming data comprises receiving a drug identifier and a concentration of a drug corresponding to the drug identifier, and wherein identifying the deviation comprises: determining that the drug is identified in a drug library stored in a memory of the infusion device; and determining that the received concentration of the drug is outside an allowable range for the concentration stored in the drug library.

Clause 7. The infusion device of any one of Clauses 1 through 6, wherein the pump controller is further configured to: receive scanned information from a medication container that was scanned by a scanner associated with the infusion device; send the scanned information to the server with a request to obtain the automated programming data, wherein the automated programming data is received after the scanned information is sent to the server; determine that the infusion device already received at least some parameters for the medication via the user interface when the automated programming data is received; and prompt via the one or more prompts, a user to confirm use of the automated programming data received from the server in place of the at least some parameters already received.

Clause 8. The infusion device of any one of Clauses 1 through 7, wherein the pump controller is further configured to: determine that the automated programming data is for changing a parameter of the infusion of the medication while the infusion is being administered to a patient, wherein identifying the deviation comprises determining, when the automated programming data is received, that the infusion is not being administered to the patient; and after determining that the infusion is not being administered, prompt via the one or more user prompts, a user to start or continue administering the infusion to the patient.

Clause 9. The infusion device of any one of Clauses 1 through 8, wherein the pump controller is further configured to: resolve a plurality of deviations for a plurality of automated programming data received from the server; identify a common resolution to a common deviation of the plurality of deviations based on a plurality of user responses to user prompts for resolving the deviations; receive, by the infusion device, new automated programming data for a new infusion to be administered by the infusion device; identify the common deviation in the new automated programming data; and automatically resolve the common deviation in the new automated programming data based on the identified common resolution.

Clause 10. The infusion device of any one of Clauses 1 through 9, wherein identifying the deviation comprises: determine that the automated programming data is for a drug to be administered to a patient after a first drug is administered to the patient; prompt via the one or more prompts, a user to program the infusion device to administer the first drug; and after the infusion device is programmed to administer the first drug, automatically load parameters corresponding to the automated programming data from the stored programming data.

Clause 11. A method performed by an infusion device, comprising: receiving, from a server remote from the infusion device, automated programming data configured to instruct the infusion device regarding an infusion of a medication; identifying a deviation in the received automated programming data with respect to stored programming data; sending, without rejecting the received automated programming data, an acknowledgement of the automated programming data and the deviation; display a user interface workflow for correcting the deviation; providing, via a user interface displayed on a display of the infusion device, one or more user prompts according to the determined user interface workflow to resolved the deviation; determining whether the deviation has been resolved; if the deviation is resolved then activating the infusion; and if the deviation is not resolved then sending an indication to the server regarding the deviation.

Clause 12. The method of Clause 11, wherein receiving the automated

programming data comprises receiving a drug identifier and a request to load parameters for a drug corresponding to the drug identifier; wherein identifying the deviation comprises determining that the drug is not identified in a currently active first drug library stored in a memory of the infusion device.

Clause 13. The method of Clause 12, further comprising: determining, by the infusion device, that the parameters to be loaded for drug are stored in a deactivated second drug library stored in the memory of the infusion device; and prompting via the one or more prompts, a user to confirm use of the second drug library; and based on receiving a confirmation to use the second drug library, deactivating the currently active first drug library, activating the second drug library, and loading the parameters for the drug from the activated second drug library.

Clause 14. The method of Clause 13, wherein the infusion device comprises a first pump channel and a second pump channel, the method further comprising: identifying the first pump channel for receiving the automated programming data; determining that the parameters to be loaded are more aligned with the second pump channel than with the first pump channel; and prompting via the one or more prompts, a user to confirm use of the second pump channel for receiving the automated programming data.

Clause 15. The method of Clause 13 or Clause 14, further comprising: determining that the second drug library is for a different care area than a care area currently activated for the infusion device.

Clause 16. The method of any one of Clauses 11 through 15, wherein receiving the automated programming data comprises receiving a drug identifier and a concentration of a drug corresponding to the drug identifier, wherein identifying the deviation comprises: determining that the drug is identified in a drug library stored in a memory of the infusion device; and determining that the received concentration of the drug is outside an allowable range for the concentration stored in the drug library.

Clause 17. The method of any one of Clauses 11 through 16, further comprising: receiving scanned information from a medication container that was scanned by a scanner associated with the infusion device; sending the scanned information to the server with a request to obtain the automated programming data, wherein the automated programming data is received after the scanned information is sent to the server; determining that the infusion device already received at least some parameters for the medication via the user interface when the automated programming data is received; and prompting via the one or more prompts, a user to confirm use of the automated programming data received from the server in place of the at least some parameters already received.

Clause 18. The method of any one of Clauses 11 through 17, further comprising: determining that the automated programming data is for changing a parameter of the infusion of the medication while the infusion is being administered to a patient, wherein identifying the deviation comprises determining, when the automated programming data is received, that the infusion is not being administered to the patient; and after determining that the infusion is not being administered, prompting via the one or more user prompts, a user to start or continue administering the infusion to the patient.

Clause 19. The method of any one of Clauses 11 through 18, further comprising: resolving a plurality of deviations for a plurality of automated programming data received from the server; identifying a common resolution to a common deviation of the plurality of deviations based on a plurality of user responses to user prompts for resolving the deviations; receiving, by the infusion device, new automated programming data for a new infusion to be administered by the infusion device; identifying the common deviation in the new automated programming data; and automatically resolving the common deviation in the new automated programming data based on the identified common resolution.

Clause 20. The method of any one of Clauses 11 through 19, wherein identifying the deviation comprises: determining that the automated programming data is for a drug to be administered to a patient after a first drug is administered to the patient; prompting via the one or more prompts, a user to program the infusion device to administer the first drug; and after the infusion device is programmed to administer the first drug, automatically loading parameters corresponding to the automated programming data from the stored programming data.

Clause 21. A non-transitory computer-readable medium comprising instructions that, when executed by a processor, cause an infusion device to perform the method of any one of Clauses 11-20.

Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™,.NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. An infusion device, comprising:
a display device; and
a pump controller comprising a processor;
wherein the pump controller is configured to:
receive, from a server remote from the infusion device, automated programming data configured to instruct the infusion device regarding an infusion of a medication;
identify a deviation in the received automated programming data with respect to stored programming data;
send, without rejecting the received automated programming data, an acknowledgement of the automated programming data and the deviation;
display a user interface workflow for correcting the deviation;
provide, via a user interface displayed on the display device, one or more user prompts according to the determined user interface workflow to resolved the deviation;
determine whether the deviation has been resolved;
if the deviation is resolved then activate the infusion; and
if the deviation is not resolved then send an indication to the server regarding the deviation.

2. The infusion device of Claim 1,
wherein receiving the automated programming data comprises receiving a drug identifier and a request to load parameters for a drug corresponding to the drug identifier; and
wherein identifying the deviation comprises determining that the drug is not identified in a currently active first drug library stored in a memory of the infusion device.

3. The infusion device of Claim 2, wherein the pump controller is further configured to:
determine, by the infusion device, that the parameters to be loaded for drug are stored in a deactivated second drug library stored in the memory of the infusion device;
prompt via the one or more prompts, a user to confirm use of the second drug library; and
based on receiving a confirmation to use the second drug library, deactivate the currently active first drug library, activate the second drug library, and load the parameters for the drug from the activated second drug library.

4. The infusion device of Claim 3, wherein the infusion device comprises a first pump channel and a second pump channel, wherein the pump controller is further configured to:
identify the first pump channel for receiving the automated programming data;
determine that the parameters to be loaded are more aligned with the second pump channel than with the first pump channel; and
prompt via the one or more prompts, a user to confirm use of the second pump channel for receiving the automated programming data.

5. The infusion device of Claim 3, wherein the pump controller is further configured to:
determine that the second drug library is for a different care area than a care area currently activated for the infusion device.

6. The infusion device of Claim 1, wherein receiving the automated programming data comprises receiving a drug identifier and a concentration of a drug corresponding to the drug identifier, and wherein identifying the deviation comprises:
determining that the drug is identified in a drug library stored in a memory of the infusion device; and
determining that the received concentration of the drug is outside an allowable range for the concentration stored in the drug library.

7. The infusion device of Claim 1, wherein the pump controller is further configured to:
receive scanned information from a medication container that was scanned by a scanner associated with the infusion device;
send the scanned information to the server with a request to obtain the automated programming data, wherein the automated programming data is received after the scanned information is sent to the server;
determine that the infusion device already received at least some parameters for the medication via the user interface when the automated programming data is received; and
prompt via the one or more prompts, a user to confirm use of the automated programming data received from the server in place of the at least some parameters already received.

8. The infusion device of Claim 1, wherein the pump controller is further configured to:
determine that the automated programming data is for changing a parameter of the infusion of the medication while the infusion is being administered to a patient, wherein identifying the deviation comprises determining, when the automated programming data is received, that the infusion is not being administered to the patient; and
after determining that the infusion is not being administered, prompt via the one or more user prompts, a user to start or continue administering the infusion to the patient.

9. The infusion device of Claim 1, wherein the pump controller is further configured to:
resolve a plurality of deviations for a plurality of automated programming data received from the server;
identify a common resolution to a common deviation of the plurality of deviations based on a plurality of user responses to user prompts for resolving the deviations;
receive, by the infusion device, new automated programming data for a new infusion to be administered by the infusion device;
identify the common deviation in the new automated programming data; and
automatically resolve the common deviation in the new automated programming data based on the identified common resolution.

10. The infusion device of Claim 1, wherein identifying the deviation comprises:
determine that the automated programming data is for a drug to be administered to a patient after a first drug is administered to the patient;
prompt via the one or more prompts, a user to program the infusion device to administer the first drug; and
after the infusion device is programmed to administer the first drug, automatically load parameters corresponding to the automated programming data from the stored programming data.

11. A method performed by an infusion device, comprising:
receiving, from a server remote from the infusion device, automated programming data configured to instruct the infusion device regarding an infusion of a medication;
identifying a deviation in the received automated programming data with respect to stored programming data;
sending, without rejecting the received automated programming data, an acknowledgement of the automated programming data and the deviation;
display a user interface workflow for correcting the deviation;
providing, via a user interface displayed on a display of the infusion device, one or more user prompts according to the determined user interface workflow to resolved the deviation;
determining whether the deviation has been resolved;
if the deviation is resolved then activating the infusion; and
if the deviation is not resolved then sending an indication to the server regarding the deviation.

12. The method of Claim 11,
wherein receiving the automated programming data comprises receiving a drug identifier and a request to load parameters for a drug corresponding to the drug identifier; and
wherein identifying the deviation comprises determining that the drug is not identified in a currently active first drug library stored in a memory of the infusion device;

13. The method of Claim 12, further comprising:
determining, by the infusion device, that the parameters to be loaded for drug are stored in a deactivated second drug library stored in the memory of the infusion device; and
prompting via the one or more prompts, a user to confirm use of the second drug library; and
based on receiving a confirmation to use the second drug library, deactivating the currently active first drug library, activating the second drug library, and loading the parameters for the drug from the activated second drug library.

14. The method of Claim 13, wherein the infusion device comprises a first pump channel and a second pump channel, the method further comprising:
identifying the first pump channel for receiving the automated programming data;
determining that the parameters to be loaded are more aligned with the second pump channel than with the first pump channel; and
prompting via the one or more prompts, a user to confirm use of the second pump channel for receiving the automated programming data.

15. The method of Claim 13, further comprising:
determining that the second drug library is for a different care area than a care area currently activated for the infusion device.

16. The method of Claim 1, wherein receiving the automated programming data comprises receiving a drug identifier and a concentration of a drug corresponding to the drug identifier, wherein identifying the deviation comprises:
determining that the drug is identified in a drug library stored in a memory of the infusion device; and
determining that the received concentration of the drug is outside an allowable range for the concentration stored in the drug library.

17. The method of Claim 11, further comprising:
receiving scanned information from a medication container that was scanned by a scanner associated with the infusion device;
sending the scanned information to the server with a request to obtain the automated programming data, wherein the automated programming data is received after the scanned information is sent to the server;
determining that the infusion device already received at least some parameters for the medication via the user interface when the automated programming data is received; and
prompting via the one or more prompts, a user to confirm use of the automated programming data received from the server in place of the at least some parameters already received.

18. The method of Claim 11, further comprising:
determining that the automated programming data is for changing a parameter of the infusion of the medication while the infusion is being administered to a patient, wherein identifying the deviation comprises determining, when the automated programming data is received, that the infusion is not being administered to the patient; and
after determining that the infusion is not being administered, prompting via the one or more user prompts, a user to start or continue administering the infusion to the patient.

19. The method of Claim 11, further comprising:
resolving a plurality of deviations for a plurality of automated programming data received from the server;
identifying a common resolution to a common deviation of the plurality of deviations based on a plurality of user responses to user prompts for resolving the deviations;
receiving, by the infusion device, new automated programming data for a new infusion to be administered by the infusion device;
identifying the common deviation in the new automated programming data; and
automatically resolving the common deviation in the new automated programming data based on the identified common resolution.

20. The method of Claim 11, wherein identifying the deviation comprises:
determining that the automated programming data is for a drug to be administered to a patient after a first drug is administered to the patient;
prompting via the one or more prompts, a user to program the infusion device to administer the first drug; and
after the infusion device is programmed to administer the first drug, automatically loading parameters corresponding to the automated programming data from the stored programming data.

21. A non-transitory computer-readable medium comprising instructions that, when executed by a processor, cause an infusion device to perform the method of Claim 11.
